(19) United States
(12) Patent Application Publication
(10) Pub. No.: US 2021/0145417 A1
(43) Pub. Date: May 20, 2021

(54) NEEDLE BIOPSY DEVICE

(71) Applicant: BOSTON SCIENTIFIC SCIMED, INC., Maple Grove, MN (US)

(72) Inventors: James P. ROHL, Prescott, WI (US); Patrick A. HAVERKOST, Corcoran, MN (US); Joseph MAYO, Medford, MA (US); Troy Anthony GIESE, Blaine, MN (US); Joel N. GROFF, Delano, MN (US)

(21) Appl. No.: 16/949,401

(22) Filed: Oct. 28, 2020

Related U.S. Application Data
(60) Provisional application No. 62/937,949, filed on Nov. 20, 2019.

(51) Int. Cl. A61B 10/02; A61B 10/04

(52) U.S. Cl. CPC A61B 10/0233; A61B 2010/045; A61B 10/04

(57) ABSTRACT

A device includes a hollow needle with a lumen extending therethrough. The needle is sized and shaped to extend through an endoscopic shaft to a target tissue within a living body. The needle has a distal end with a sharpened distal tip for puncturing the target tissue and removing a portion of the tissue in the lumen. The device further includes a cylindrical stylet having a shaft sized and shaped to extend through the lumen of the needle and a distal end with a pointed distal tip for puncturing the target tissue. When the stylet is extended, the pointed distal tip of the stylet extends distally a predetermined distance past the sharpened distal tip of the needle.

### PRIORITY CLAIM

The present disclosure claims priority to U.S. Provisional Patent Application Ser. No. 62/937,949 filed Nov. 20, 2019; the disclosure of which is incorporated herewith by reference.

### FIELD

The present disclosure relates to fine needle biopsy (FNB) devices with improved puncturing qualities.

### BACKGROUND

Fine needle biopsies are often performed under endoscopic ultrasound (EUS) guidance to collect core tissue samples (biopsies) for evaluation. After the target anatomy, e.g. a lesion, has been visualized using EUS, a sheathed fine needle biopsy (FNB) device is advanced to the target anatomy to puncture the lesion capsule and acquire tissue in the lumen of the hollow needle.

Various mechanical and biological constraints may cause difficulty in puncturing the target anatomy. For example, dense or hardened areas near or within the target anatomy, e.g., gastrointestinal stromal tumors (GISTs) or pancreatic calcifications, may deflect the needle into surrounding, non-targeted tissue during an attempted puncture. In another example, the target anatomy may be reachable only from a shallow approach angle, causing the needle to slip along an outer surface of the lesion rather than puncturing the target structure.

### SUMMARY

The present disclosure relates to a device including a hollow needle with a lumen extending therethrough, the needle being sized and shaped to extend through an endoscopic shaft to a target tissue within a living body, the needle having a distal end with a sharpened distal tip for puncturing the target tissue and removing a portion of the tissue in the lumen; and a cylindrical stylet having a shaft sized and shaped to extend through the lumen of the needle and a distal end with a pointed distal tip for puncturing the target tissue. when the stylet is extended, the pointed distal tip of the stylet extends distally a predetermined distance past the sharpened distal tip of the needle.

In an embodiment, the distal end of the stylet has a tapered ogival profile.

In an embodiment, the stylet shaft is closely fitted to the lumen of the needle when the stylet is extended therethrough.

In an embodiment, the predetermined distance the pointed distal tip of the stylet extends distally past the sharpened distal tip of the needle corresponds to a length of the tapered distal end of the stylet.

In an embodiment, the distal end of the needle has a Franseen grind with three pointed tips separated from one another circumferentially by three ground notches.

In an embodiment, the needle is formed from a cobalt-chromium alloy.

In an embodiment, the stylet is formed from a nitinol alloy. The present disclosure also relates to a device including a hollow needle with a lumen extending therethrough, the needle being sized and shaped to extend through an endoscopic shaft to a target tissue within a living body, the needle having a distal end with a sharpened distal tip for puncturing the target tissue and removing a portion of the tissue in the lumen; a hollow cylindrical dilator with a lumen extending therethrough and having a shaft sized and shaped to extend through the lumen of the needle; and a wire sized and shaped to extend through the lumen of the dilator and having a puncturing tip for puncturing the target tissue.

In an embodiment, the cylindrical dilator has a rounded distal end with an atraumatic distal tip.

In an embodiment, when the dilator is extended distally out the distal end of the needle and the wire is extended distally out the distal end of the dilator, the dilator extends a first predetermined distance past the sharpened distal tip of the needle and the wire extends a second predetermined distance past the atraumatic distal tip of the dilator.

In an embodiment, the wire is advanceable distally out the distal end of the dilator and retractable thereinto via a spring-loaded push button on a handle of the device.

In an embodiment, the rounded distal end of the dilator is adhered the dilator shaft.

In an embodiment, the rounded distal end of the dilator is formed from a polymer, the dilator shaft is formed from a braided or coiled polymer composite and the puncturing wire is formed from nitinol.

In an embodiment, the distal end of the needle has a Franseen grind with three pointed tips separated from one another circumferentially by three ground notches and the needle is formed from a cobalt-chromium or nitinol alloy.

In an embodiment, the dilator shaft is closely fitted to the lumen of the needle when the dilator is extended therethrough.

Furthermore, the present disclosure relates to a method including extending a cylindrical stylet through a lumen of a hollow needle, the needle being sized and shaped to extend through an endoscopic shaft to a target tissue within a living body, the needle having a distal end with a sharpened distal tip for puncturing the target tissue and removing a portion of the tissue in the lumen, the stylet having a shaft sized and shaped to extend through the lumen of the needle, the stylet having a distal end with a pointed distal tip for puncturing the target tissue, the pointed distal tip of the stylet extending distally a predetermined distance past the sharpened distal tip of the needle; puncturing the target tissue with the stylet and advancing the stylet and the hollow needle distally into the target tissue; retracting the stylet proximally through the lumen of the needle; and acquiring a sample of the target tissue with the hollow needle.

In an embodiment, the distal end of the stylet has a tapered ogival profile.

In an embodiment, the stylet shaft is closely fitted to the lumen of the needle when the stylet is extended therethrough.

In an embodiment, the predetermined distance the pointed distal tip of the stylet extends distally past the sharpened distal tip of the needle corresponds to a length of the tapered distal end of the stylet.

In an embodiment, the distal end of the needle has a Franseen grind with three pointed tips separated from one another circumferentially by three ground notches, the needle being formed from a cobalt-chromium alloy.

### BRIEF DESCRIPTION OF THE DRAWINGS

FIG. 1 shows an exemplary embodiment of a biopsy needle for use in an EUS-FNB procedure in accordance with the present disclosure.

FIG. 2 shows an exemplary FNB device including the biopsy needle of FIG. 1 with a stylet having a sharp bullet-nose distal tip.

FIG. 3 shows a side view the FNB device of FIG. 2.

FIG. 4 shows an exemplary FNB device including the biopsy needle 100 of FIG. 1 with a hollow dilator 300 extending therethrough in accordance with the present disclosure.

FIG. 5 shows a side-section view of the FNB device of FIG. 4.

FIG. 6 shows the FNB device of FIG. 4 with a puncturing wire extending out the hollow dilator.

FIG. 7 shows a side view of the FNB device of FIG. 4.

FIG. 8 shows an exemplary enclosure for attaching the dilator of FIG. 4 to the needle of FIG. 1 and actuating the puncturing wire.

FIG. 9 shows an exemplary biopsy needle having a distal tip with a modified Franseen grind.

### DETAILED DESCRIPTION

The present disclosure may be further understood with reference to the following description and the appended drawings, wherein like elements are referred to with the same reference numerals. The exemplary embodiments describe fine needle biopsy (FNB) needles with improved puncture performance. In some embodiments, a sharpened puncturing element extending distally out the lumen of the needle is used to puncture the target anatomical structure prior to advancing the needle into and acquiring tissue from that anatomical structure. The sharpened puncturing element may have a further dilating effect to ease the insertion of the needle into the anatomy prior to tissue acquisition, as described below. It is common practice in fine needle biopsy to acquire tissue from the core of a lesion and not solely from an exterior or capsule area of the lesion. Thus, each of the devices has means for preventing non-targeted tissue from being acquired and for preventing the acquisition of tissue from the target structure until after the needle has been advanced into the target structure to a desired puncture depth.

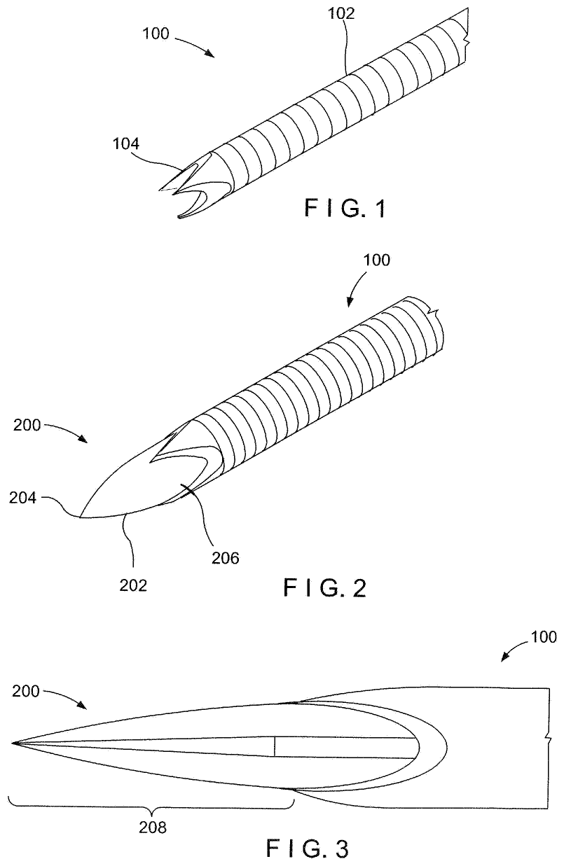

FIG. 1 shows a distal end of a biopsy needle 100 for use in an endoscopic ultrasound fine needle biopsy (EUS-FNB) procedure. The needle 100 includes a hollow shaft 102 having a distal end 104 with a sharpened distal tip for puncturing and collecting tissue from a target anatomical structure (e.g., a lesion) when introduced into the target anatomical structure via an insertion device such as, for example, a flexible endoscope. The needle 100 is, in this embodiment, formed from a cobalt-chromium (CoCr) alloy. CoCr has high strength properties, allowing needle tips fashioned from CoCr to resist bending during puncturing. However, other materials may be used for the needle 100, including, for example, nitinol or stainless steel. For example, nitinol may be used for larger gauge needles to avoid kinking. The distal end 104 in this embodiment has a Franseen grind, which results in a crown-like shape with three pointed tips separated from one another circumferentially by three ground notches. However, other shapes for the distal end 104 may be used, such as a beveled end, or any number of pointed tips.

As shown in FIGS. 2-3, an FNB device including the biopsy needle 100 of FIG. 1 further includes a stylet 200 having a bullet-nose distal end 202 with a sharp tissue piercing distal tip 204 extending from a cylindrical shaft 206, the stylet 200 being sized and shaped to extend through the lumen of the hollow shaft 102 so that, in a distal-most position, the tip 204 of the stylet 200 extends distally out of the distal end 104 of the needle 100. The distal end 202 has a tapered cross-section with a side-section profile that may be considered substantially ogival. In other words, the “bullet-nose” of this embodiment has a side-section, as seen in FIG. 3, formed by two symmetrical segments of a curve (e.g., a circle) meeting at a point, i.e., the distal tip 204. In this case, the radius of the needle 200 is smaller than a radius of each of the curves defining the symmetrical segments so that the tip 202 is less blunt (more tapered) than a hemisphere having the same diameter as the needle 200.

The curve of the distal end 202 gradually and smoothly transitions to the cylindrical shape of the shaft 206 at its proximal end. The radius of the curved segment side-section may vary. For instance, an ogival distal end 202 may have a profile matching a shorter arc length of a larger radius curve, or a longer arc length of a smaller radius circle. In other embodiments, the distal end 202 may not be strictly ogival, yet have a similar taper that, at its proximal base, remains tangent to the shaft 206 and curves radially inward but more closely resembles a conical taper approaching the distal tip 204. In still other embodiments, the segment of the curve may be elliptical.

The stylet 200 in this embodiment is formed from a superelastic nitinol alloy permitting the stylet/needle combination to navigate a tortuous path along the way to the target tissue through tight turning radii without plastic deformation. Other flexible alloys may be used as well as would be understood by those skilled in the art. The length of the stylet 200 is selected so that, when inserted to a distal-most position within the needle 100, the distal tip 204 of the tapered distal end 202 extends a predetermined desired distance 208 (a “setback”) distally out of the distal end 104 of the needle 100, as shown in FIG. 3. The FNB device may be configured so that the setback 208 corresponds to the distance from the distal tip 204 to the flat of the shaft 206 of the stylet 200. In other words, the setback 208 may correspond to the length of the bullet-nose distal tip 202, which may vary based on the gauge of the needle 100. The setback 208 is dependent on the diameter of the needle 100 with a range of the setback 208 being approximately 0.08″-0.6″. In one embodiment, the setback 208 for the stylet 200 is −0.1″. In alternate embodiments, where a needle having a differently shaped tip (e.g., beveled tip) is used, a similar configuration for the setback 208 can be used. The stylet 200 is shaped so that, even at shallow approach angles, the distal tip 204 lodges in the target tissue. For example, the distal tip 204 of the stylet 200 may effectively puncture tissue at angles of 5° or more.

After puncturing the tissue, the stylet 200 may be further used to dilate the target capsule. The bullet nose shape of the distal end 202 of the stylet 200 (i.e., the gradual increase in the diameter of the distal end 202 moving proximally from a minimum at the distal tip 204 thereof) serves to spread the tissue as the stylet 200 is advanced distally into the tissue so that the needle 100 may be more smoothly inserted into the lesion behind the stylet 200. As noted previously, some EUS-FNB procedures are used to acquire tissue from a core of a lesion.

For these procedures, it is not desirable to begin acquiring tissue until after the needle 100 has penetrated the lesion to a desired depth. To this end, the stylet shaft 206 is sized to minimize a clearance (i.e. the annular gap) between the stylet 200 and the inner diameter of the hollow shaft 102, while remaining slidable therein, so that, as the stylet 200 is maintained in its distal-most position covering the distal opening of the hollow shaft 102, no tissue enters the needle during the puncturing of the lesion. After the needle 100 has been inserted into the lesion to a sufficient depth, the stylet 200 is withdrawn proximally from the needle 100 and the needle 100 is advanced further distally into the lesion to acquire a core tissue sample. If multiple samples are to be taken, the stylet 200 may again be inserted through the needle 100 and operated in a similar manner. In addition, if any prong on the tip 104 of the needle 100 was bent in any prior tissue acquisitions, the stylet 200, being closely fitted with an inner diameter (ID) of the needle 100, will straighten the bend(s) the next time it is advanced through the distal end 104.

In another embodiment to be described below, a wire is advanced distally out of the distal end of a hollow dilator 300 to facilitate the initial puncturing of the target tissue via a controlled actuation from the needle handle. The wire may be spring-loaded or non-spring-loaded, and be actuated via a push button, slider, trigger, or some other actuator.

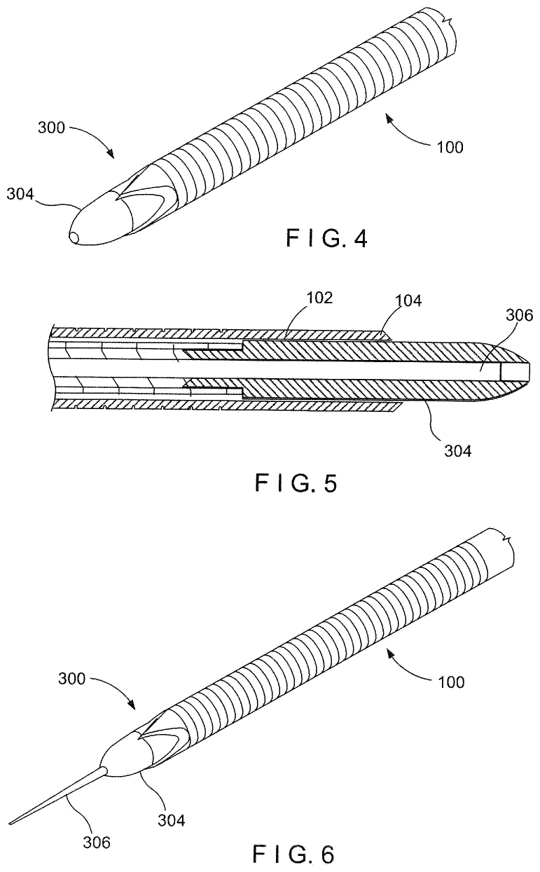

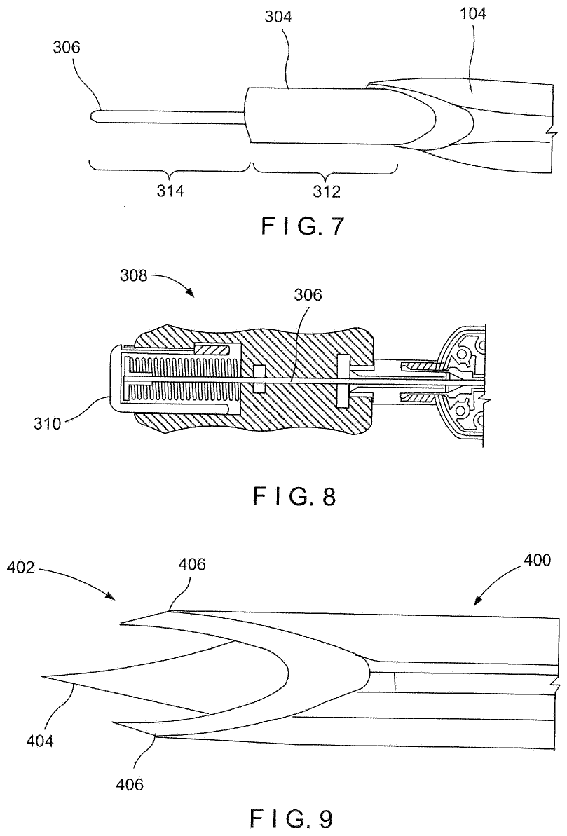

As shown in FIGS. 4-7, an FNB device according to a further embodiment includes a biopsy needle 100 as described above in regard to FIG. 1 with a dilator 300 received in a lumen of the needle 100. The dilator 300 includes a shaft 302 with a lumen extending therethrough. The dilator 300 extends to a rounded distal tip 304 with a distal opening through which a puncturing wire 306 may be advanced out of the dilator lumen to project distally from a distal end of the dilator 300. FIGS. 4-5 show the dilator 300 with the puncturing wire 306 retracted within the lumen, i.e., in a pre-actuated state, while FIGS. 6-7 show the puncturing wire 306 extending distally out of the dilating tip 304, i.e., in an actuated state. The dilator shaft 302 may be formed, for example, from a superelastic material such as nitinol tubing or a polymer composite-sheathed coil or braid, while the dilating tip 304 may be a suitable biocompatible metal (such as nitinol), a polymer (e.g., PEEK, polycarbonate), glass, etc., as would be understood by those skilled in the art. The material of the dilating tip 304 may be selected to adhere well to the shaft 302 behind it.

The puncturing wire 306 may also be formed of a superelastic material such as, for example, nitinol. The diameter of the lumen of the dilator 300, and the corresponding diameter of the puncturing wire 306, are selected to be small enough so that the wire 306 is capable of puncturing a target lesion even if the distal tip is not separately machined to enhance a sharpness of the distal tip. For example, the diameter may be 0.006″. The distal tip of the wire 306 may also be sharpened to a point or have a wedged trocar tip. The setback 312 between the dilating tip 304 and the distal end 104 of the needle may be configured similarly to that described above with respect to the stylet 200, i.e., may correspond to the distance from the distal-most point of the distal tip 304 of the dilator 300 to the flat of the dilator shaft 302. The setback 314 between the wire 306 and the dilating tip 304 may be a variable length.

For example, the wire 306 may have a shorter setback that functions similarly to the bullet-nose distal end 202 of the stylet 200 when the wire 306 is extended. Considered this way, the combination of the dilator 300 and the wire 306 provides an alternative to the stylet 200 that has a rounded, i.e. atraumatic, tip until the operating physician actuates the wire 306. In another embodiment, the wire 306 may have a longer setback relative to the dilating tip 304. In this embodiment, the wire 306 may be used to reach out and engage the lesion, even at very shallow approach angles. For example, the wire 306 may initially engage the lesion at the shallow approach angle and bend slightly to facilitate advancing the remainder of the device (dilator 300, needle 100) into the lesion.

The dilator 300, including the puncturing wire 306, extends from an enclosure 308, as shown in FIG. 8. The enclosure 308 may, for example, be threaded onto a luer of a needle handle and, in this embodiment, has a push button 310 for deploying the wire 306, i.e., extending the wire 306 from the pre-actuated state to the actuated state. However, actuators other than the push button 310 may be used. The deployment of the wire 306 may be rapid, or it may be slow.

For example, when the wire 306 is in the pre-actuated state, the spring may be compressed so that, when the actuator is operated, the spring is released to drive the wire 306 rapidly distally out of the distal end of the dilator 300 to penetrate target tissue. That is, when it is desired to penetrate a target anatomical structure, the distal end of the dilator 300 is placed adjacent the desired puncture site and the actuator is operated to drive the wire 306 distally out of the dilator 300 into the target tissue.

In an alternate embodiment, the wire 306 is advanced at any pace (fast or slow), and the tip of the wire 306 is then brought adjacent to the tissue for puncturing. The user may then advance the dilator 300 and the needle 100 distally over the wire 306 into the target tissue mass. Once the needle has been advanced into the target tissue mass to a desired depth, the wire 306 and the dilator 300 may be withdrawn proximally (either retracted proximally to a desired distance within the needle 100 or fully withdrawn therefrom) and the needle 100 may be advanced further into the target tissue mass to capture a tissue sample within the needle 100.

As described above, during an EUS-FNB procedure, the enclosure 308 is coupled to the needle 100, as described above, and the dilating tip 304 is brought to a desired position adjacent to the target anatomy. The push button 310 is then actuated, extending the tip of the puncturing wire 306 distally out of the distal end of the dilating tip 304 so that the wire 306 punctures the target tissue. The user then advances dilator 300 and the needle 100 distally into the target tissue with the gradual increase in the diameter of the dilator 300 from a minimum at its distal end, gradually spreading open an opening formed by the wire 306 to facilitate a smooth entry of the needle 100 into the target tissue mass in a manner similar to that described in regard to the dilating stylet 200 with respect to FIG. 2. After the needle has been advanced into the target tissue mass to a desired depth, the dilator 300 is withdrawn proximally and the needle 100 is advanced distally to acquire the core tissue.

FIG. 9 shows a biopsy needle 400 having a distal tip 402 with modified Franseen grind that may be used in place of the needle 100 in the same manner described above with either the stylet 200 or a hollow dilator 300 and wire 306. Instead of the three equal-sized puncturing prongs shown with respect to the needle 100 shown in FIG. 1, the biopsy needle 400 has a prong 404 extending to a longer axial reach (i.e., extending further distally) than the other two of the prongs 406. The long prong 404 extends distally beyond distal ends of the other prongs 406 to allow the needle 400 to achieve an initial anchoring in a target tissue mass, providing stability as the rest of the tip 402 is advanced into the lesion. If desired, the needle 400 may perform the initial puncturing and no stylet, or a blunt stylet may be used with the needle 400. A blunt stylet provides inner diameter support when the needle 400 takes a tortuous path, as well as protecting the tips of the Franseen grind during advancement of the needle 400. Further, the blunt stylet may prevent the distal tip 402 from damaging the endoscope as the needle 400 is advanced distally therethrough. However, once the tip 402 has been advanced past the distal end of the endoscope, the blunt stylet may be withdrawn.

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this invention is not limited to the particular embodiment disclosed, but rather modifications are also covered within the scope of the present invention as defined by the appended claims. Specifically, although this application describes various embodiments each having different features in various combinations, those skilled in the art will understand that any of the features of one embodiment may be combined with the features of the other embodiments in any manner not specifically disclaimed or which is not functionally or logically inconsistent with the operation of the device or the stated functions of the disclosed embodiments.

1-15. (canceled)

16. A device, comprising:
a hollow needle with a lumen extending therethrough, the needle being sized and shaped to extend through an endoscopic shaft to a target tissue within a living body, the needle having a distal end with a sharpened distal tip for puncturing the target tissue and removing a portion of the tissue in the lumen; and
a cylindrical stylet having a shaft sized and shaped to extend through the lumen of the needle and a distal end with a pointed distal tip for puncturing the target tissue,
wherein, when the stylet is extended, the pointed distal tip of the stylet extends distally a predetermined distance past the sharpened distal tip of the needle.

17. The device of claim 16, wherein the distal end of the stylet has a tapered ogival profile.

18. The device of claim 17, wherein the stylet shaft is closely fitted to the lumen of the needle when the stylet is extended therethrough.

19. The device of claim 18, wherein the predetermined distance the pointed distal tip of the stylet extends distally past the sharpened distal tip of the needle corresponds to a length of the tapered distal end of the stylet.

20. The device of claim 16, wherein the distal end of the needle has a Franseen grind with three pointed tips separated from one another circumferentially by three ground notches.

21. The device of claim 16, wherein the needle is formed from a cobalt-chromium alloy.

22. The device of claim 16, wherein the stylet is formed from a nitinol alloy.

23. A device, comprising:
a hollow needle with a lumen extending therethrough, the needle being sized and shaped to extend through an endoscopic shaft to a target tissue within a living body, the needle having a distal end with a sharpened distal tip for puncturing the target tissue and removing a portion of the tissue in the lumen;
a hollow cylindrical dilator with a lumen extending therethrough and having a shaft sized and shaped to extend through the lumen of the needle; and
a wire sized and shaped to extend through the lumen of the dilator and having a puncturing tip for puncturing the target tissue.

24. The device of claim 23, wherein the cylindrical dilator has a rounded distal end with an atraumatic distal tip.

25. The device of claim 24, wherein, when the dilator is extended distally out the distal end of the needle and the wire is extended distally out the distal end of the dilator, the dilator extends a first predetermined distance past the sharpened distal tip of the needle and the wire extends a second predetermined distance past the atraumatic distal tip of the dilator.

26. The device of claim 25, wherein the wire is advanceable distally out the distal end of the dilator and retractable thereinto via a spring-loaded push button on a handle of the device.

27. The device of claim 24, wherein the rounded distal end of the dilator is adhered the dilator shaft.

28. The device of claim 27, wherein the rounded distal end of the dilator is formed from a polymer, the dilator shaft is formed from a braided or coiled polymer composite and the puncturing wire is formed from nitinol.

29. The device of claim 23, wherein the distal end of the needle has a Franseen grind with three pointed tips separated from one another circumferentially by three ground notches and the needle is formed from a cobalt-chromium or nitinol alloy.

30. The device of claim 23, wherein the dilator shaft is closely fitted to the lumen of the needle when the dilator is extended therethrough.

31. A method, comprising:
extending a cylindrical stylet through a lumen of a hollow needle, the needle being sized and shaped to extend through an endoscopic shaft to a target tissue within a living body, the needle having a distal end with a sharpened distal tip for puncturing the target tissue and removing a portion of the tissue in the lumen, the stylet having a shaft sized and shaped to extend through the lumen of the needle, the stylet having a distal end with a pointed distal tip for puncturing the target tissue, the pointed distal tip of the stylet extending distally a predetermined distance past the sharpened distal tip of the needle;
puncturing the target tissue with the stylet and advancing the stylet and the hollow needle distally into the target tissue;
retracting the stylet proximally through the lumen of the needle; and
acquiring a sample of the target tissue with the hollow needle.

32. The method of claim 31, wherein the distal end of the stylet has a tapered ogival profile.

33. The method of claim 32, wherein the stylet shaft is closely fitted to the lumen of the needle when the stylet is extended therethrough.

34. The method of claim 33, wherein the predetermined distance the pointed distal tip of the stylet extends distally past the sharpened distal tip of the needle corresponds to a length of the tapered distal end of the stylet.

35. The method of claim 31, wherein the distal end of the needle has a Franseen grind with three pointed tips separated from one another circumferentially by three ground notches, the needle being formed from a cobalt-chromium alloy.
